(19) United States
(12) Patent Application Publication
(10) Pub. No.: US 2025/0114314 A1
(43) Pub. Date: Apr. 10, 2025

(54) COMPOSITION AND METHOD FOR TREATING MENSTRUATION RELATED SYMPTOMS

(71) Applicant: Margo Harrison, Denver, CO (US)

(72) Inventor: Margo Harrison, Denver, CO (US)

(21) Appl. No.: 18/376,910

(22) Filed: Oct. 5, 2023

(51) Int. Cl. A61K 31/192; A61K 31/047; A61K 31/138; A61K 31/167; A61K 31/225; A61K 31/29; A61K 31/341; A61K 31/355; A61K 31/4045; A61K 31/426; A61K 31/4402; A61K 31/4415; A61K 31/451; A61K 31/51; A61K 31/593; A61K 31/7016; A61K 31/717; A61K 31/765; A61K 31/80; A61K 33/06; A61K 33/08; A61K 33/10; A61K 33/26; A61K 33/30; A61K 35/60; A61K 36/35; A61K 36/48; A61K 36/68; A61K 36/9068

(52) U.S. Cl. CPC A61K 31/192; A61K 31/047; A61K 31/138; A61K 31/167; A61K 31/225; A61K 31/29; A61K 31/341; A61K 31/355; A61K 31/4045; A61K 31/426; A61K 31/4402; A61K 31/4415; A61K 31/451; A61K 31/51; A61K 31/593; A61K 31/7016; A61K 31/717; A61K 31/765; A61K 31/80; A61K 33/06; A61K 33/08; A61K 33/10; A61K 33/26; A61K 33/30; A61K 35/60; A61K 36/35; A61K 36/48; A61K 36/68; A61K 36/9068

(57) ABSTRACT

The present invention discloses a composition and method for treating menstruation related symptoms. The composition comprises an effective amount of a nonsteroidal anti-inflammatory drug (NSAID) comprising at least one of naproxen and ketoprofen. The composition is administrated before an onset of menstruation or during menstruation. The composition is provided in a pharmaceutical package. The package comprises a label indicating a quantity of the formulation to be consumed for a specific time and day in coordination with a menstrual period.

### TECHNICAL FIELD

The present invention generally relates to treatment of menstruation related symptoms. More specifically, the present invention relates to composition and method for treating menstruation related symptoms.

### BACKGROUND

Dysmenorrhea refers to pain associated with menstruation. Generally, the menstrual cycle involves a series of hormonal changes. A consequence of these hormonal changes is the growth of the uterine lining or endometrium. The endometrium is thick with fluids and nutrients designed to nourish an embryo. If no egg has been fertilized, the top layers of the endometrium are shed and the process is called menstruation. This process involves the release of prostaglandins, which cause contractions of the smooth muscle in the uterus. In some women, these contractions cause substantial pain or dysmenorrhea, which interferes with their daily activities. More than half of women who menstruate have some pain for 1 to 2 days each month. Usually, the pain is mild. But for some women, the pain is severe. Further, for some women, discomfort may also be present in the days preceding onset of menstruation.

Treatment of dysmenorrhea is typically accomplished by rest and local application of heat. In more severe cases, analgesics and sedatives are sometimes prescribed. Further, few existing patent applications attempt to address the problems cited in the background as prior art over the presently disclosed subject matter and are explained as follows.

US20090291140 of Andrew Korey entitled “treatment of dysmenorrhea via transdermal administration of nonsteroidal anti-inflammatory drugs” discloses methods and compositions for the treatment of a subject suffering from dysmenorrhea, including both primary and secondary dysmenorrhea. The composition includes transdermal NSAID formulations. The method involves transdermally administering to the subject an effective amount of a nonsteroidal anti-inflammatory agent.

US20210113493 of Philip E. Wolfson entitled “Ketamine for the treatment of menstrually related symptoms” discloses methods, compositions, and kits for the treatment of all forms of emotional and painful symptoms related to the menstrual cycle, including symptoms characteristic of menstrual cycle-related disorders, such as premenstrual dysphoric disorder (PMDD), premenstrual syndrome (PMS), menopause, perimenopause, and sub-diagnostic emotional, psychological, cognitive, spiritual, and/or physical symptoms of the menstrual cycle. The method involves administering to the subject a composition comprising ketamine, norketamine, 6-hydroxynorketamine, or a pharmaceutically acceptable salt thereof, in an amount sufficient to treat the menstrually related symptom.

Presently, only a limited number of techniques, compositions, and methods are available for reducing the discomforting symptoms associated with menses, such as pain. Further, existing compositions and methods lack an effective solution to treat the pain and other symptoms associated with menses.

Therefore, there is a need for composition and method for treating menstruation-related symptoms.

### SUMMARY

The present invention discloses a composition and method for treating menstruation-related symptoms. The composition and method of the present invention are configured to reduce menstrual pain and bleeding. The composition comprises an effective amount of a nonsteroidal anti-inflammatory drug (NSAID) comprising at least one of naproxen and ketoprofen. In one embodiment, the present invention discloses a method for treating the menstruation related symptoms. At one step, the composition comprising an effective amount of a nonsteroidal anti-inflammatory drug (NSAID) comprising at least one of naproxen and ketoprofen is provided. At another step, the composition is administered to a subject before an onset of menstruation or during menstruation.

The composition further comprises at least one of inositol, simethicone, acetaminophen and combination thereof. The composition further comprises antacids comprising at least one of calcium carbonate, aluminium hydroxide, magnesium hydroxide, sodium bicarbonate, magnesium hydroxide, famotidine, ranitidine and combination thereof. The composition further comprises laxatives comprising at least one of psyllium husk, methylcellulose, docusate sodium, docusate calcium, polyethylene glycol, lactulose, magnesium hydroxide, sorbitol, senna, bisacodyl and combination thereof.

The composition further comprises anti-diarrheal agents comprising at least one of loperamide, bismuth subsalicylate and combination thereof. The composition further comprises anti-fungal agents.

The composition further comprises at least one of vitamin E, calcium, vitamin B1, vitamin B6, fish oil, vitamin D3, ginger powder, iron, magnesium, zinc and combination thereof. The composition further comprises sleep aid agents comprising at least one of diphenhydramine, doxylamine, melatonin, valerian root and combination thereof.

In one embodiment, the composition is provided in the form of pills. In one embodiment, the composition is provided in a pharmaceutical package. The package includes a label indicating a quantity of the formulation to be consumed for a specific time and day in coordination with a menstrual period. In one embodiment, the pharmaceutical package comprises a blister package, boxes, cartons, sachets, tubes, pumps, ampules, vials, dropper bottles, pouches, chewable pouches, and dissolvable strips.

In one embodiment, a method for treating one or more symptoms of premenstrual syndrome of an individual, is also disclosed. The method is performed by administering a composition comprising: naproxen or ketoprofen with a list of over-the-counter (OTC) medications and supplements at least one of: inositol, simethicone, acetaminophen and combination thereof; at least one of calcium carbonate, aluminium hydroxide, magnesium hydroxide, sodium bicarbonate, magnesium hydroxide, famotidine, ranitidine and combination thereof, and laxatives comprising at least one of psyllium husk, methylcellulose, docusate sodium, docusate calcium, polyethylene glycol, lactulose, magnesium hydroxide, sorbitol, senna, bisacodyl and combination thereof; anti-diarrheal agents comprising at least one of loperamide, bismuth subsalicylate and combination thereof; anti-fungal agents; at least one of vitamin E, calcium, vitamin B1, vitamin B6, fish oil, vitamin D3, ginger powder, iron, magnesium, zinc and combination thereof, and sleep aid agents comprising at least one of diphenhydramine, doxylamine, melatonin, valerian root and combination thereof. The compositions housed within the packaging consist of a metal or plastic foil, typically arranged in a blister pack format. This packaging not only contains the composition but also features comprehensive instructions for its appropriate administration. The recommended timing for administering the composition to an individual is approximately one, two, three, four, or five days prior to the onset of menses. In another aspect, the recommended timing for administering the composition to an individual is one or two days prior to the onset of menses.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 exemplarily illustrates a block diagram of a composition for treating menstruation related symptoms, according to an embodiment of the present invention.

FIG. 2 exemplarily illustrates a flowchart of a method for treating menstruation related symptoms, according to an embodiment of the present invention.

FIG. 3 exemplarily illustrates a table of composition comprising naproxen and information related to dosage of composition to be administered, according to an embodiment of the present invention.

FIG. 4 exemplarily illustrates a table of composition comprising Vitamin E and information related to dosage of composition to be administered, according to another embodiment of the present invention.

FIG. 5 exemplarily illustrates a table of composition comprising naproxen and iron, and information related to dosage of composition to be administered, according to yet another embodiment of the present invention.

FIG. 6 exemplarily illustrates a table of composition comprising naproxen and simethicone, and information related to dosage of composition to be administered, according to yet another embodiment of the present invention.

FIG. 7 exemplarily illustrates a table of composition comprising naproxen and inositol, and information related to dosage of composition to be administered, according to yet another embodiment of the present invention.

FIG. 8 exemplarily illustrates a table of composition comprising naproxen and acetaminophen, and information related to dosage of composition to be administered, according to yet another embodiment of the present invention.

FIG. 9 exemplarily illustrates a table of composition comprising vitamin E and melatonin, and information related to dosage of composition to be administered, according to yet another embodiment of the present invention.

FIG. 10 exemplarily illustrates a table of composition comprising naproxen and melatonin, and information related to dosage of composition to be administered, according to yet another embodiment of the present invention.

FIG. 11 exemplarily illustrates a flowchart of a method to treat primary dysmenorrhea, according to yet another embodiment of the present invention.

### DETAILED DESCRIPTION OF EXAMPLE EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

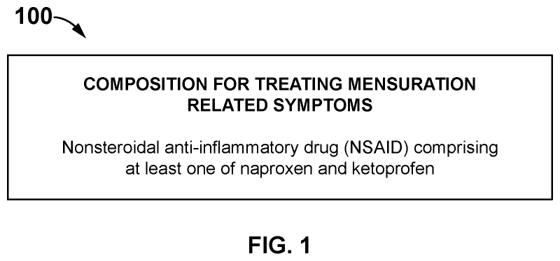

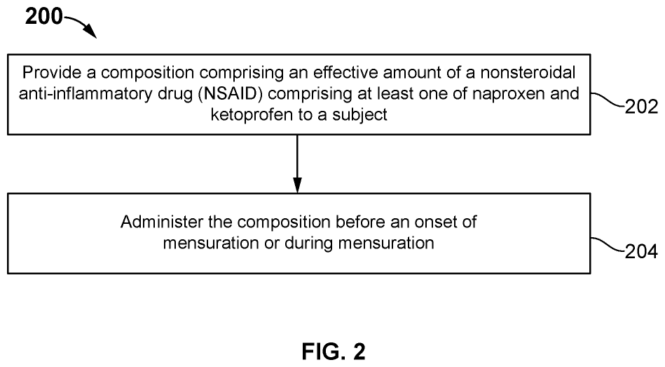

Referring to FIG. 1 and FIG. 2, the present invention discloses a composition 100 and method 200 for treating menstruation related symptoms. The composition 100 comprises an effective amount of a nonsteroidal anti-inflammatory drug (NSAID). In one embodiment, the NSAID comprises naproxen. In another embodiment, the NSAID comprises ketoprofen.

The composition 100 further comprises at least one of inositol, simethicone, acetaminophen and combination thereof. In one embodiment, the Naproxen could be used in combination with other drugs including but not limited to inositol, simethicone, acetaminophen.

The composition 100 further comprises antacids comprising at least one of calcium carbonate, aluminium hydroxide, magnesium hydroxide, sodium bicarbonate, magnesium hydroxide, famotidine, ranitidine and combination thereof. The composition 100 further comprises laxatives comprising at least one of psyllium husk, methylcellulose, docusate sodium, docusate calcium, polyethylene glycol, lactulose, magnesium hydroxide, sorbitol, senna, bisacodyl and combination thereof.

The composition 100 further comprises anti-diarrheal agents comprising at least one of loperamide, bismuth subsalicylate and combination thereof. The composition 100 further comprises anti-fungal agents. The composition 100 further comprises at least one of vitamin E, calcium, vitamin B1, vitamin B6, fish oil, vitamin D3, ginger powder, iron, magnesium, zinc and combination thereof. The composition 100 further comprises sleep aid agents comprising at least one of diphenhydramine, doxylamine, melatonin, valerian root and combination thereof.

In one embodiment, the composition 100 is provided in the form of pills. In one embodiment, the composition 100 is provided in a pharmaceutical package. The package comprises a label indicating a quantity of the formulation to be consumed for a specific time and day in coordination with a menstrual period. The compositions presented in the package made of metal or plastic foil. In one embodiment, the pharmaceutical package comprises blister packages boxes, cartons, sachets, tubes, pumps, ampules, vials, dropper bottles, pouches, chewable pouches and dissolvable strips. The package comprises instructions for administration.

In one embodiment, the composition 100 comprising ketoprofen and the composition 100 comprising naproxen are provided in the same blister package. In another embodiment, the composition 100 comprising ketoprofen and the composition 100 comprising naproxen are provided in a separate blister package. In yet another embodiment, the composition 100 comprising ketoprofen and the composition 100 comprising naproxen are provided in a sachet or other methods of delivering the medication.

Referring to FIG. 2, the present invention discloses a method 200 for treating the menstruation related symptoms. At step 202, the composition 100 comprising an effective amount of a nonsteroidal anti-inflammatory drug (NSAID) comprising at least one of naproxen and ketoprofen is provided. At step 204, the composition 100 is administered to a subject before an onset of menstruation or during menstruation.

In accordance with the method 200 of the present invention, the composition 100 further comprises at least one of inositol, simethicone, acetaminophen and combination thereof. The composition 100 further comprises antacids comprising at least one of calcium carbonate, aluminium hydroxide, magnesium hydroxide, sodium bicarbonate, magnesium hydroxide, famotidine, ranitidine and combination thereof. The composition 100 further comprises laxatives comprising at least one of psyllium husk, methylcellulose, docusate sodium, docusate calcium, polyethylene glycol, lactulose, magnesium hydroxide, sorbitol, senna, bisacodyl and combination thereof.

The composition 100 further comprises anti-diarrheal agents comprising at least one of loperamide, bismuth subsalicylate and combination thereof. The composition 100 further comprises anti-fungal agents.

The composition 100 further comprises at least one of vitamin E, calcium, vitamin B1, vitamin B6, fish oil, vitamin D3, ginger powder, iron, magnesium, zinc and combination thereof. The vitamin E and iron of the composition 100 could treat anemia. Further, vitamin E could also reduce menstrual pain and bleeding. The composition 100 further comprises sleep aid agents comprising at least one of diphenhydramine, doxylamine, melatonin, valerian root and combination thereof.

Further, the composition 100 is also allowed for intake under the FDA label. For example, initially, 500 mg could be taken once, at the onset of menses, or 1 to 2 days prior to onset of menses for severe symptoms. Thereafter, 500 mg could be consumed for every 12 hours as needed or 250 mg every 6 to 8 hours as needed. For example, for a duration of one to five days, the composition 100 of 1.25 g/day on day 1, and thereafter 1 g/day could be consumed for treating the menstruation related symptoms.

In one embodiment, a method for treating one or more symptoms of premenstrual syndrome of an individual, is also disclosed. The method is performed by administering a composition comprising: naproxen or ketoprofen with a list of over-the-counter (OTC) medications and supplements at least one of: inositol, simethicone, acetaminophen and combination thereof; at least one of calcium carbonate, aluminium hydroxide, magnesium hydroxide, sodium bicarbonate, magnesium hydroxide, famotidine, ranitidine and combination thereof, and laxatives comprising at least one of psyllium husk, methylcellulose, docusate sodium, docusate calcium, polyethylene glycol, lactulose, magnesium hydroxide, sorbitol, senna, bisacodyl and combination thereof; anti-diarrheal agents comprising at least one of loperamide, bismuth subsalicylate and combination thereof; anti-fungal agents; at least one of vitamin E, calcium, vitamin B1, vitamin B6, fish oil, vitamin D3, ginger powder, iron, magnesium, zinc and combination thereof, and sleep aid agents comprising at least one of diphenhydramine, doxylamine, melatonin, valerian root and combination thereof. The compositions housed within the packaging consist of a metal or plastic foil, typically arranged in a blister pack format. This packaging not only contains the composition but also features comprehensive instructions for its appropriate administration. The recommended timing for administering the composition to an individual is approximately one, two, three, four, or five days prior to the onset of menses. In another embodiment, the recommended timing for administering the composition to an individual is one or two days prior to the onset of menses.

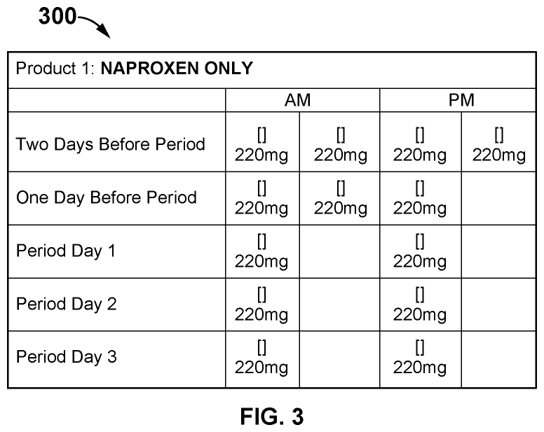

FIG. 3 exemplarily illustrates a table 300 of composition comprising naproxen and information related to dosage of composition to be administered, according to an embodiment of the present invention.

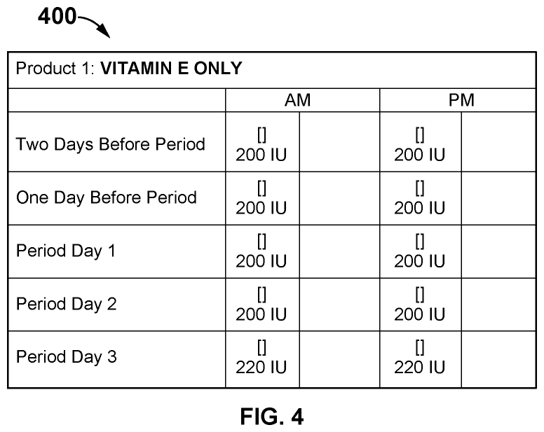

FIG. 4 exemplarily illustrates a table 400 of composition comprising Vitamin E and information related to dosage of composition to be administered, according to another embodiment of the present invention.

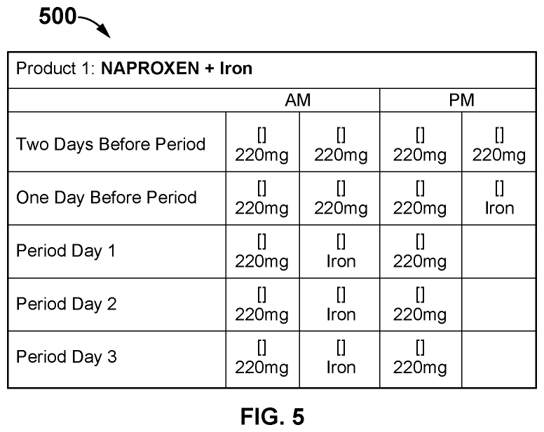

FIG. 5 exemplarily illustrates a table 500 of composition comprising naproxen and iron, and information related to dosage of composition to be administered, according to yet another embodiment of the present invention.

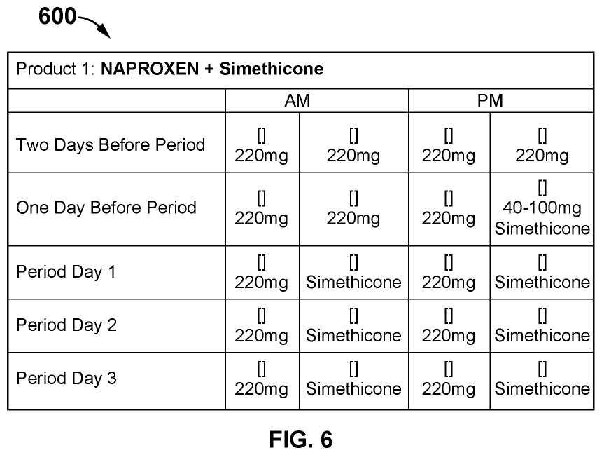

FIG. 6 exemplarily illustrates a table 600 of composition comprising naproxen and simethicone, and information related to dosage of composition to be administered, according to yet another embodiment of the present invention.

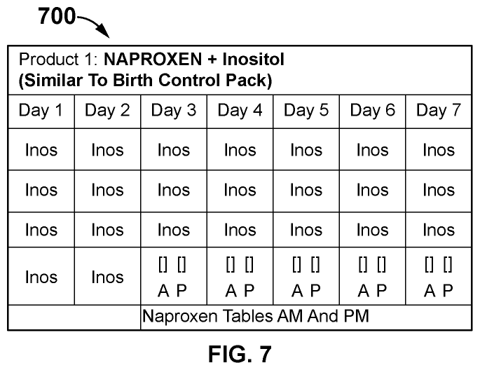

FIG. 7 exemplarily illustrates a table 700 of composition comprising naproxen and inositol, and information related to dosage of composition to be administered, according to yet another embodiment of the present invention. In one embodiment, magnesium could be added to the melatonin. In another embodiment, B vitamins, Diphenhydramine and senna could be added to the composition.

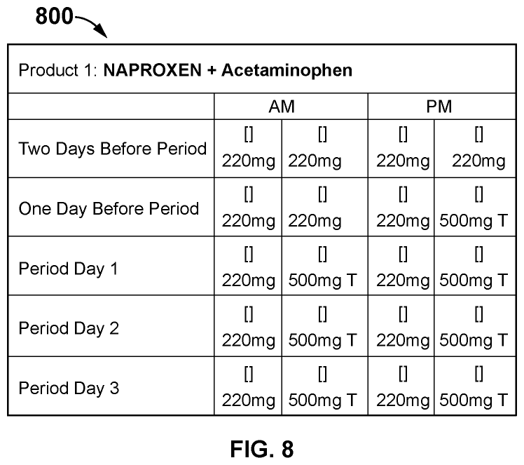

FIG. 8 exemplarily illustrates a table 800 of composition comprising naproxen and acetaminophen, and information related to dosage of composition to be administered, according to yet another embodiment of the present invention.

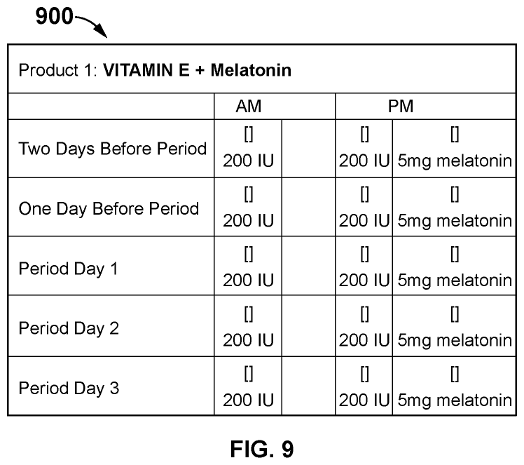

FIG. 9 exemplarily illustrates a table 900 of composition comprising vitamin E and melatonin, and information related to dosage of composition to be administered, according to yet another embodiment of the present invention.

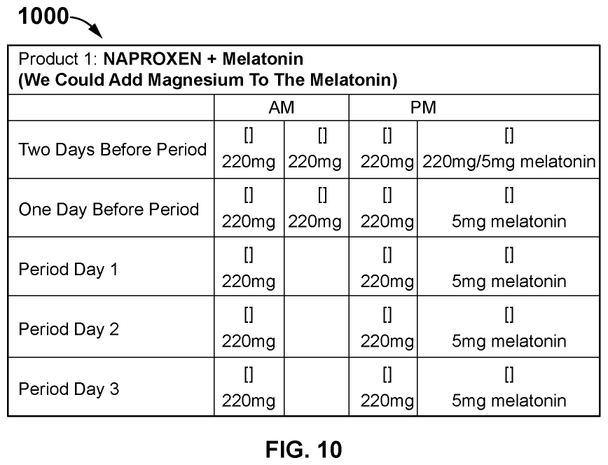

FIG. 10 exemplarily illustrates a table 1000 of composition comprising naproxen and melatonin, and information related to dosage of composition to be administered, according to yet another embodiment of the present invention.

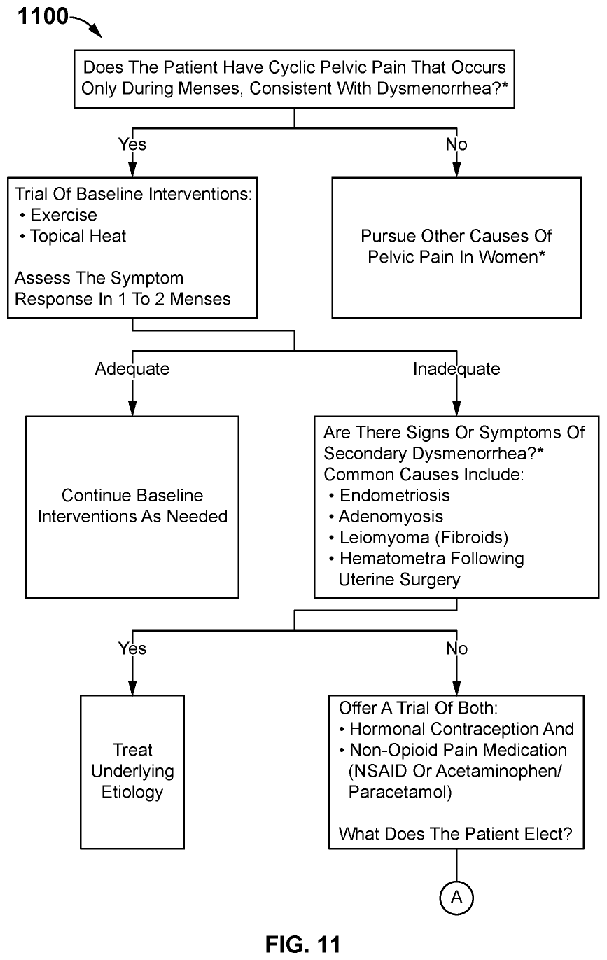

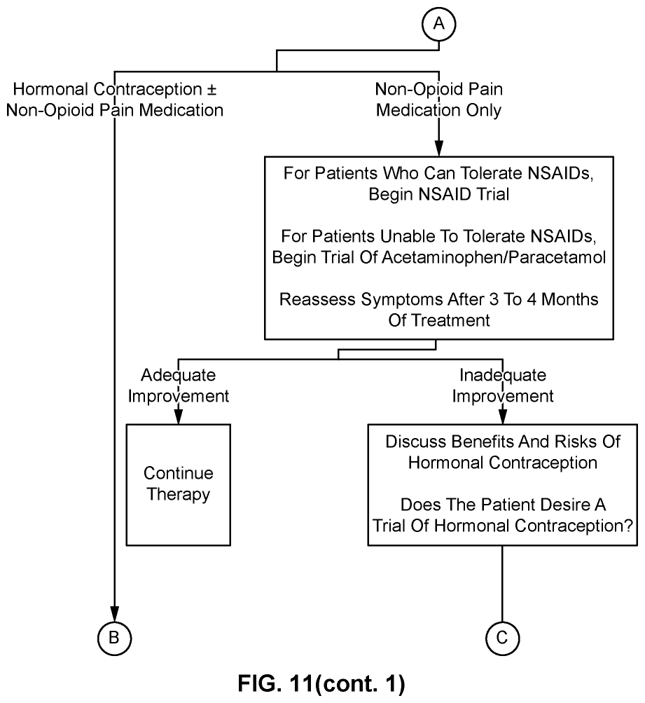

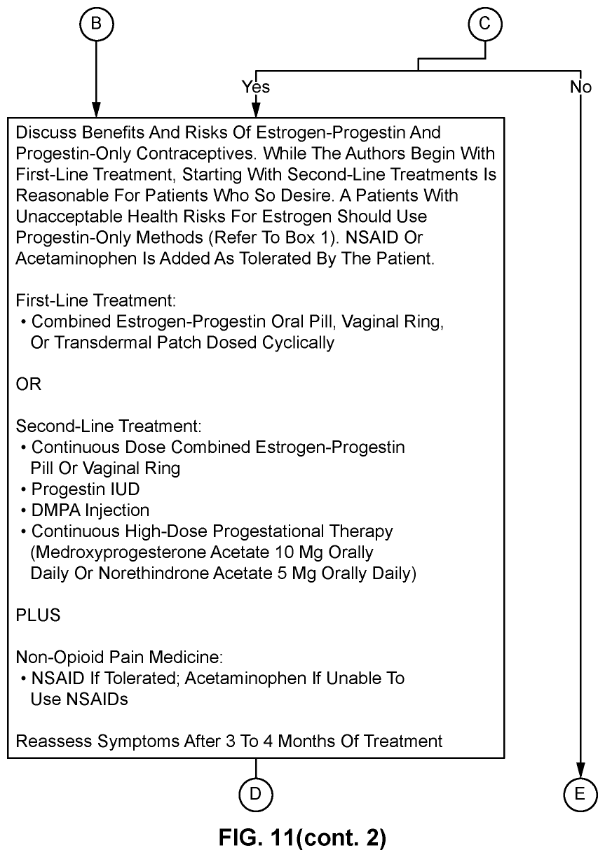

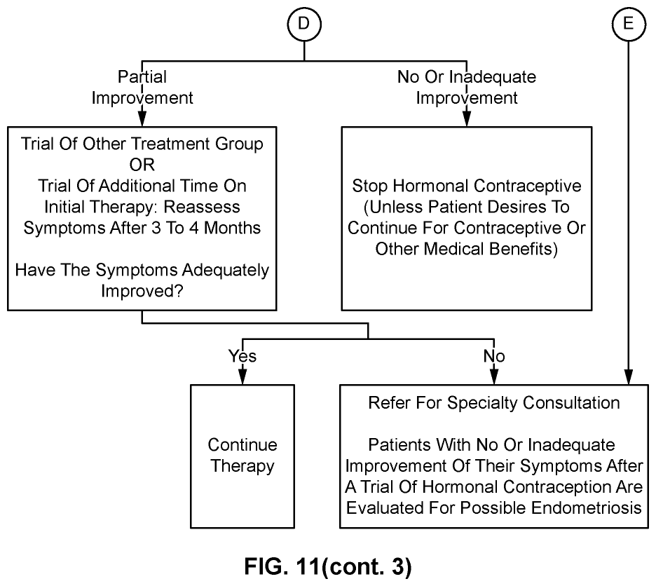

FIG. 11 exemplarily illustrates a flowchart of a method 1100 to treat primary dysmenorrhea, according to yet another embodiment of the present invention. The method 1100 provides an approach to treat primary dysmenorrhea in adult women. If baseline intervention such as exercise and topical heat fails in treatment of primary dysmenorrhea, depending on the preferences of the patient and the health condition of the patient, non-opioid pain medication, hormonal contraception, or combination of both are used to treat primary dysmenorrhea.

The composition 100 and method 200 of the present invention are configured to reduce menstrual pain and bleeding. The composition 100 is configured to treat primary dysmenorrhea. The composition 100 could be used prior to onset of menses for severe symptoms. Alternatively, the composition 100 could be used at the onset of menses. The composition 100 of the present invention is dosed and packaged for premenstrual treatment for pain and bleeding control followed by a few final days of iron to help restore Hb levels after menstruation. Further, the blister pack of pills indicates the time and days to be taken in coordination with the menstrual period. For example, the blister pack could be labelled with “two days before period” at respective AM and PM doses, “one day before period” at respective AM and PM doses. Further, for example, the label includes AM and PM doses that need to be taken three days before the menstruation period.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A composition for treating menstruation related symptoms, comprising:
an effective amount of a nonsteroidal anti-inflammatory drug (NSAID) comprising at least one of naproxen and ketoprofen.

2. The composition of claim 1, further comprises at least one of inositol, simethicone, acetaminophen and combination thereof.

3. The composition of claim 1, further comprises antacids comprising at least one of calcium carbonate, aluminium hydroxide, magnesium hydroxide, sodium bicarbonate, magnesium hydroxide, famotidine, ranitidine and combination thereof.

4. The composition of claim 1, further comprises laxatives comprising at least one of psyllium husk, methylcellulose, docusate sodium, docusate calcium, polyethylene glycol, lactulose, magnesium hydroxide, sorbitol, senna, bisacodyl and combination thereof.

5. The composition of claim 1, further comprises anti-diarrheal agents comprising at least one of loperamide, bismuth subsalicylate and combination thereof.

6. The composition of claim 1, further comprises anti-fungal agents.

7. The composition of claim 1, further comprises at least one of vitamin E, calcium, vitamin B1, vitamin B6, fish oil, vitamin D3, ginger powder, iron, magnesium, zinc and combination thereof.

8. The composition of claim 1, further comprises sleep aid agents comprising at least one of diphenhydramine, doxylamine, melatonin, valerian root and combination thereof.

9. The composition of claim 1, is provided in a form of pills.

10. The composition of claim 1, is provided in a pharmaceutical package, wherein the package comprises a label indicating a quantity of the composition to be consumed for a specific time and day in coordination with a menstrual period.

11. The composition of claim 1, wherein the pharmaceutical package comprises a blister package, boxes, cartons, sachets, tubes, pumps, ampules, vials, dropper bottles, pouches, chewable pouches and dissolvable strips.

12. A method for treating menstruation related symptoms, comprising the steps of:
providing a composition comprising an effective amount of a nonsteroidal anti-inflammatory drug (NSAID) comprising at least one of naproxen and ketoprofen to a subject, and
administering the composition before an onset of menstruation or during menstruation.

13. The method of claim 12, wherein the composition further comprises at least one of inositol, simethicone, acetaminophen and combination thereof.

14. The method of claim 12, wherein the composition further comprises antacids comprising at least one of calcium carbonate, aluminium hydroxide, magnesium hydroxide, sodium bicarbonate, magnesium hydroxide, famotidine, ranitidine and combination thereof, and laxatives comprising at least one of psyllium husk, methylcellulose, docusate sodium, docusate calcium, polyethylene glycol, lactulose, magnesium hydroxide, sorbitol, senna, bisacodyl and combination thereof.

15. The method of claim 12, wherein the composition further comprises anti-diarrheal agents comprising at least one of loperamide, bismuth subsalicylate and combination thereof.

16. The method of claim 12, wherein the composition further comprises anti-fungal agents.

17. The method of claim 12, wherein the composition further comprises at least one of vitamin E, calcium, vitamin B1, vitamin B6, fish oil, vitamin D3, ginger powder, iron, magnesium, zinc and combination thereof.

18. The method of claim 12, wherein the composition further comprises sleep aid agents comprising at least one of diphenhydramine, doxylamine, melatonin, valerian root and combination thereof.

19. A method for treating one or more symptoms of premenstrual syndrome of an individual by administering a composition comprising naproxen or ketoprofen with a list of over-the-counter (OTC) medications and supplements at least one of: inositol, simethicone, acetaminophen and combination thereof; at least one of calcium carbonate, aluminium hydroxide, magnesium hydroxide, sodium bicarbonate, magnesium hydroxide, famotidine, ranitidine and combination thereof, and laxatives comprising at least one of psyllium husk, methylcellulose, docusate sodium, docusate calcium, polyethylene glycol, lactulose, magnesium hydroxide, sorbitol, senna, bisacodyl and combination thereof; anti-diarrheal agents comprising at least one of loperamide, bismuth subsalicylate and combination thereof; anti-fungal agents; at least one of vitamin E, calcium, vitamin B1, vitamin B6, fish oil, vitamin D3, ginger powder, iron, magnesium, zinc and combination thereof, and sleep aid agents comprising at least one of diphenhydramine, doxylamine, melatonin, valerian root and combination thereof.
